(19) United States
(12) Patent Application Publication
(10) Pub. No.: US 2010/0071703 A1
(43) Pub. Date: Mar. 25, 2010

(54) CONDOM HOLDER

(76) Inventor: Alexander Leonidovich Felitsyn, Saint Petersburg (RU)

Correspondence Address: Maria Nilova;Patentica, Sadovaya Street, 51, Office 303, Saint Petersburg, 190068, RU

(21) Appl. No.: 12/517,072

(22) PCT Filed: Dec. 26, 2007
(86) PCT No.: PCT/RU07/00758

§ 371 Date: Jun. 1, 2009

(51) Int. Cl. A61F 6/04; B25B 3/00

(52) U.S. Cl. 128/844; 269/2

(57) ABSTRACT

The condom holder according to the invention comprises fasteners for fixing the condom edge, and a connection between said fasteners; said connection at least partially being elastic, the holder at least partially defining a space in which the condom can be placed; the holder being adapted to change the shape of said connection between a first configuration in which said space is reduced, and a second configuration in which said space is expanded. In the first said configuration at least part of said connection is strained and tends to transform into the second configuration, and in the second configuration said strain is partially released.

Due to the fact that the holder is elastic, the condom placed therein is automatically stretched as required, there is no need to control the force applied by the user. The condom can be put on using only one hand holding the holder. Further, the possibility of excessive or insufficient expansion of the holder is excluded.

### CROSS-REFERENCE TO RELATED APPLICATIONS

This application claims the benefits from International Application PCT/RU2007/000758 filed on Dec. 26, 2007 and priority application RU 2006147417 filed on Dec. 29, 2006. The contents of these applications are hereby incorporated by reference and in their entirety.

### FIELD OF THE INVENTION

The present invention relates to barrier contraceptives, in particular to a condom holder.

### BACKGROUND OF THE INVENTION

A condom is a skin made of natural latex or another material, covering the penis entirely or partially. Condoms are used to protect from sexually transmitted diseases and/or undesirable pregnancy. A traditional condom is shown in FIG. 1. A condom can also be a so-called micro-condom covering only the balanus.

To make it compact and easy to use, a condom is usually rolled into ring. Actually, it can also be rolled in an opposite direction to that traditionally used for rolling a condom. A condom can also be bellowed or otherwise folded. When stored, a condom is usually sealingly packed into a skin generally formed as a package of an elastic sheet material which is usually strong enough to provide tightness and protect the condom during storage and transportation (see FIG. 2). A package is usually provided with means facilitating its unpacking, e.g. a notch or reduced-strength area at its side, or a sealed ribbon with one its end extending outside the package. When unpacked, a condom is applied to erected penis and then rolled down to the penis root.

It can be unnecessary to unroll down a micro-condom because it covers only the balanus. Therefore, a micro-condom only requires a minimal degree of unrolling, if any.

The main disadvantage of traditional condoms is that it takes a long time and much effort to unpack and put on a condom. As a result, the user's attention is distracted away from sexual intercourse.

It is also necessary to use both hands to put the condom on. However, typically, the user has only one free hand, and uses the other one to support himself or has it under woman's head (see FIG. 3). Movements of this hand are therefore restricted. Generally, this position is uncomfortable, and the user can release his second hand only for a short time. Therefore, for unpacking and putting on the condom, the user can use freely only one of his hands, occasionally using his second hand.

When putting on a condom, every moment spent can be critical for the user. Requirements for swift, convenient and easy handling of condom package are much higher than those for any other packed product used in private life. Besides, it is known that some people do not use condoms even when necessary, and the main reason for this is that they have to spend a long time and much effort to put a condom on, resulting in distraction of their minds from the sexual intercourse. The object is to simplify and reduce the number of steps required to unpack and put on a condom. If this problem is solved,

a) the overall condom usage will increase; whereas

b) the number of people who either do not use condoms at all, or use them rarely while having risky sexual intercourses, will decrease.

This could help to solve the spreading HIV problem as well as decrease the uncontrolled birth rate growth (especially in overpopulated developing countries).

Devices known as condom holders hold a condom, in most cases hold the edge of a rolled condom, and therefore allow the user grasp a condom holder and put the condom on by applying the condom on top of and down the penis (see e.g. FIG. 4). In this process, the condom is unrolled along the penis shaft (where a pre-rolled condom is used).

A condom holder can be a hollow ring with a groove extending along its circumference and defining a recess for receiving a condom. The shape and location of the groove can vary (e.g., see U.S. Pat. No. 4,738,357, Martin et al, 1988).

When the inner diameter of the holder is greater than or equal to the inner diameter of the condom ring, the recess can open to the outside. In this case, forces arise directed to the center of the condom ring, which do not let this ring pop out of the groove.

This kind of holder is disclosed in the following patents: JP6154260 (Toshihiro, 1994), JP7308336, Tadashi, 1995), U.S. Pat. No. 5,579,784 (Harari, 1996), DE19641107 (Raabe, 1998), U.S. Pat. No. 5,437,286 (Stratton, 1995), FR2751869 (Loyer, 1998).

Also known are condom holders either embracing the whole condom ring (e.g. see U.S. Pat. No. 5,163,449, van der Valk 1992) or having one discontinuity, either C-shaped or U-shaped (e.g. see U.S. Pat. No. 4,875,491, Parrone, 1989). Holders can either hold the condom ring with spaced fasteners (e.g. see FR2727858, Vegega, 1996) or continuously, along the whole circumference of the ring.

Other means of holding the edge of a rolled condom can be used, e.g. the condom ring can be held by a pair of rollers (e.g. see WO9801095, Liehs, 1999).

However, the above-mentioned holders touch the penis when putting on the condom, which is uncomfortable for the user. Thus, a need arises for a holder that keeps the condom stretched, and its fasteners do not touch or only slightly touch the penis.

FIG. 5 shows the holder consisting of two parts which are to be held with both hands (e.g. see WO02/069861, Van Rensburg, 2002). In this case, the fasteners are spaced from the penis because the user stretches the condom by the parts of the holder.

Also known is a two-part condom holder (WO2007/021184, Tunovic Adnan et al, 2007), each part comprising at least one fastener in the form of a loop used for fixing the condom edge from the outside. Here, the fasteners contact the penis through the condom, thus providing significantly more comfort for the user in comparison with the case where the fasteners hold the condom edge from inside and therefore touch the penis. In use, however, the condom is to be stretched by the holder to a certain extent, to avoid the fasteners overpress the penis.

According to WO2007/021184, the two parts are pivotably joined together on one end so as to swing open when putting on the condom. More specifically, the two parts are joined by a hinge member of a flexible material. The hinge member can act like a spring tending to keep the two parts closed. Each part has on its outside a gripping member to be held with two fingers. That is, while spacing apart these parts the user has to hold each gripping member with two fingers of one hand. This step is uncomfortable for the user and requires some extra skill.

Another modification of a condom holder stretching the condom ring is known from WO2006/025755 (Mor Maty Seck, 2006). The holder includes a ring with a semi-ring placed on it, and a radial guideway. A handle inserted into the guideway, is provided with another semi-ring on the side facing the ring. Both semi-rings are provided with a groove used to receive the edge of a condom rolled into a ring.

When putting on the condom held by the holder, the ring with the guideway is held in one hand, and the handle is displaced along the guideway with another hand, thus enlarging the inner area formed by two semi-rings and therefore stretching the condom ring as appropriate.

However, due to the fact that condom ring is closed, the ring can only removed in the reverse direction along the penis after putting the condom on, and this is uncomfortable for the user. Furthermore, with this holder the condom is stretched with both hands, and this is uncomfortable for the user due to the aforementioned reasons.

A holder that seems to be the closest to the one claimed herein is described in WO9700652 (Patrick, 1997). The holder includes fasteners for fastening the edge of the condom, and an elastic connection between these fasteners. The holder defines a space in which a condom can be placed. Further, the holder is adapted to change the shape of said connection between a first configuration in which said space is reduced, and a second configuration in which said space is expanded. The holder is curvilineal (spiral-shaped), and ends thereof are overlapping and provided with handles.

When putting on the condom held by described holder, the handles are displaced towards each other, thus increasing the space defined by the holder to a required degree therefore providing the comfortable process of putting the condom on.

However, when putting on the condom held by holder and subsequently removing the holder, the user must constantly act the holder, surpassing its spring resistance and keeping it in expanded state. The force applied must be measured, because the overly strong push will cause the excessive stretching of the condom, which may lead to its tearing or to the increase of the friction between the condom and the holder; the isufficiently strong push, on the other hand, will not provide sufficient widening of the holder and may cause friction between the holder and the penis. Said need for force measurement distracts the user from sexual intercourse. Furthermore, due to the overlapping ends of the holder, said holder forms a closed ring which can only be removed after putting the condom on by displacing said ring in opposite longitudinal direction, which is uncomfortable for the user.

The purpose of this invention is to provide a durable and compact condom holder which would provide fast and comfortable stretching of the condom and, subsequently, fast and comfortable process of putting on the condom. It is preferrable that all actions performed while stretching and putting on the condom are performed using one hand, and it is possible to remove the holder in lateral direction after the condom is put on; therefore, the process of putting on the condom causes only minimal distraction of the user from sexual intercourse.

### BRIEF SUMMARY OF THE INVENTION

The problem is solved by providing a condom holder comprising fasteners for fixing the edge of a condom, and a connection between these fasteners; said connection at least partially being elastic, the holder at least partially defining a space in which the condom can be placed; the holder being adapted to change the shape of said connection between a first configuration in which said space is reduced, and a second configuration in which said space is expanded. In the first said configuration at least part of said connection is strained and tends to transform into the second configuration, and in the second configuration said strain at least partially is released.

Due to the fact that the holder is elastic, the condom placed therein is automatically stretched as required, there is no need to control the force applied by the user; the condom can be put on using only one hand holding the holder. Further, excessive or insufficient widening of the holder is excluded.

The connection can also include at least one arc-shaped member and at least one connector connecting the arc-shaped members.

The arc-shaped member can be elastic.

The connector can be elastic.

The distance between the fasteners in the second configuration can be larger than the distance between the fasteners in the first configuration.

The distance between the ends of the arc-shaped member in the second configuration can be larger than the distance between the ends of the arc-shaped member in the first configuration.

Said ability to change the shape of the holder can be provided by at least one spring.

The spring can comprise an elastic wire.

The spring can be formed with coils at the middle part thereof.

The holder can include an axis disposed in the middle part of the holder, and the spring can be mounted on said axis with its coils embracing said axis.

The holder can include grooves in which both ends of the spring are placed.

The spring can be a compression spring, extension spring, flexure spring or torsion spring.

The holder can include another spring.

Said ability of changing the shape of the connection between the fasteners can be provided by at least one elastic connector connecting the arc-shaped members.

Said ability of changing the shape of the connection between the fasteners can be provided by a hinge connecting the arc-shaped members.

Said ability of changing the shape of the connection between the fasteners can be provided by a hinge connecting the arc-shaped members and by elastic webs in the arc-shaped members.

The holder can be provided with a handle on the outer side of the arc-shaped member.

The holder can be provided with another handle; both handles being arranged in such a way that when the handles are acted, the shape of the holder is changed.

The handles can be provided with a fixing device for fixing the holder in the second configuration.

The holder can be provided with a retaining device for retaining the holder in the first configuration.

The retaining device for retaining the holder in the first configuration can include a ring embracing the holder.

The holder can be at least partially received in a package.

The package can be provided with at least one weakened line.

The holder can be provided with a case into which the package with the holder received therein is placed, the package being attached to the case.

The retaining device for retaining the holder in the first configuration can be a box having one open end, wherein the holder is received in the box.

The box can include a slot, and the holder can be provided with a key connected to the holder means of a part received in said slot and adapted to displace; wherein said key is outside the box.

The holder can be provided with a fixing device for fixing the holder in the second configuration.

The holder can be provided with a retaining device for retaining the holder in the first configuration and a fixing device for fixing the holder in the second configuration.

The holder can be received in a box with a slot, the retaining device f for retaining the holder in the first configuration can include a ring embracing the holder; and the holder can be provided with a key connected to the holder by means of a part received in said slot and adapted to displace; wherein the key can be outside the box.

Said ring can be moveably connected to the box, and provided with a device preventing the ring from leaving the box when displacing the holder in the condom plane.

The problem is also solved by providing a condom holder for holding a flatly arranged condom, the holder including fasteners for fixing the condom edge, and a connection between these fasteners; said connection at least partially being elastic, the holder at least partially defining a space in which the condom can be placed; the holder being adapted to deform between a first configuration in which said space is reduced, and a second configuration in which said space is expanded. In the first said configuration at least part of said connection is strained and tends to transform into the second configuration, and in the second configuration the condom is stretched, and said strain is partially released and balanced by elastic forces resulting from the stretch of the condom.

### BRIEF DESCRIPTION OF THE DRAWINGS

Below is a detailed description of the invention with reference to the attached drawings, in which:

FIG. 1 is perspective view of a traditional condom held in hand by the user;

FIG. 2 is a perspective view of a traditional condom shown in FIG. 1, in the package;

FIG. 3 illustrates the situation preceding the process of putting on the condom;

FIG. 4 is a perspective view of a condom placed in a known ring-like holder held in hand by the user;

FIG. 5 is a perspective view of a condom placed in a known holder held with two hands by the user;

FIG. 6 is a perspective view of the condom holder in its another embodiment;

FIG. 7 is a perspective view of the holder shown in FIG. 6, with a condom installed therein;

FIG. 8 is a perspective view of the holder with condom, as shown in FIGS. 6-7, with a retaining device in one of its embodiments;

FIG. 9 is a perspective view of the holder with the condom held, as shown in FIGS. 6-7, wherein the retaining device is removed;

FIG. 10 is a perspective view of the holder with condom, as shown in FIGS. 6-7, and another embodiment of the retaining device;

FIG. 11 is a perspective view of the holder with condom, wherein the retaining device shown in FIG. 10 is removed;

FIG. 12 is a perspective view of the holder shown in FIG. 10, received in a case;

FIG. 13 is a perspective view of the holder with condom, as shown in FIG. 12, after unpacking;

FIG. 14 is a perspective view of another embodiment of the case.

FIGS. 15-16 are exploded views of the case as shown in FIG. 14, and the holder received in a package;

FIG. 17 is a perspective view of the case with the holder received therein, as shown in FIGS. 14-16, after unpacking;

FIG. 18 is a perspective view of another embodiment of the condom holder according to the invention;

FIG. 19 is an exploded view showing the holder shown in FIG. 18;

FIG. 20 is a perspective view of another embodiment of the condom holder;

FIG. 21 is a perspective view of another embodiment of the holder;

FIG. 22 is a perspective view of another embodiment of the suggested holder with condom;

FIG. 23 is an exploded view of another embodiment of the holder shown in FIG. 22;

FIG. 24 is a perspective view of another embodiment of the holder with condom;

FIG. 25 is a perspective view of another embodiment of the holder with condom;

FIG. 26 is a perspective view of the holder shown in FIG. 25, received in a package;

FIG. 27 is a perspective view of the holder shown in FIG. 26, received in a case;

FIG. 28 is a perspective view of the holder shown in FIG. 25, during the unpacking process;

FIG. 29 is a perspective view of the holder shown in FIG. 25, after unpacking;

FIG. 30 is a perspective view showing another embodiment of the holder received in the case;

FIG. 31 is a exploded view of the holder and case shown in FIG. 29;

FIGS. 32-33 illustrate the process of unpacking the holder shown in FIG. 30;

FIGS. 34-35 are cutaway views of the holder in the case, as shown in FIGS. 32-33.

FIG. 36 is a perspective view of another embodiment of the holder received in the case;

FIGS. 37-38 illustrate the process of unpacking the holder shown in FIG. 36;

FIGS. 39-40 are cutaway views of the connection between the key and the holder shown in FIGS. 37, 38.

FIG. 41 is a perspective view of another embodiment of the case;

FIG. 42 illustrates a top view of the case (lid removed) shown in FIG. 40, with the holder with condom;

FIG. 43 illustrates a top view of the case (lid removed) shown in FIG. 41, wherein the condom is unpacked.

FIGS. 44-45 are perspective views of the another embodiment of the condom holder according to the invention;

FIG. 46 is a perspective view of the another embodiment of the condom holder;

FIG. 47 is the perspective view of another embodiment of the condom holder;

FIGS. 48-49 are the perspective views of another embodiment of the holder with condom;

FIG. 50 is a perspective view of another embodiment of the condom holder;

FIGS. 51-53 are exploded views of the holder with condom in the embodiment shown in FIG. 50, with the retaining device;

FIG. 54 is a perspective view of another embodiment of the holder received in a case;

FIG. 55 is the perspective view of another embodiment of the holder shown in FIG. 54, wherein the lid is removed;

FIGS. 56-57 illustrate the process of unpacking the holder shown in FIG. 50;

FIG. 58 is a sectional view of the holder shown in FIG. 50, received in a case;

FIG. 59 is a perspective view of another embodiment of the holder received in a case; and

FIG. 60 illustrates a perspective view of another embodiment of the holder.

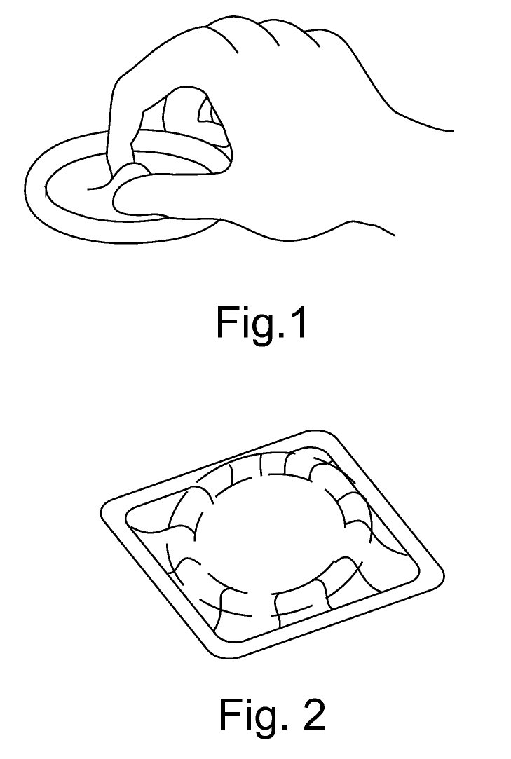

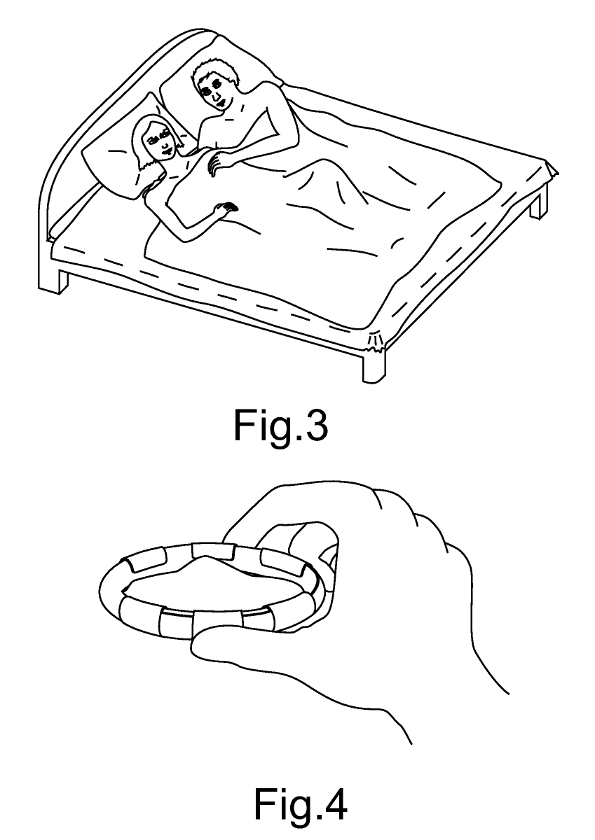

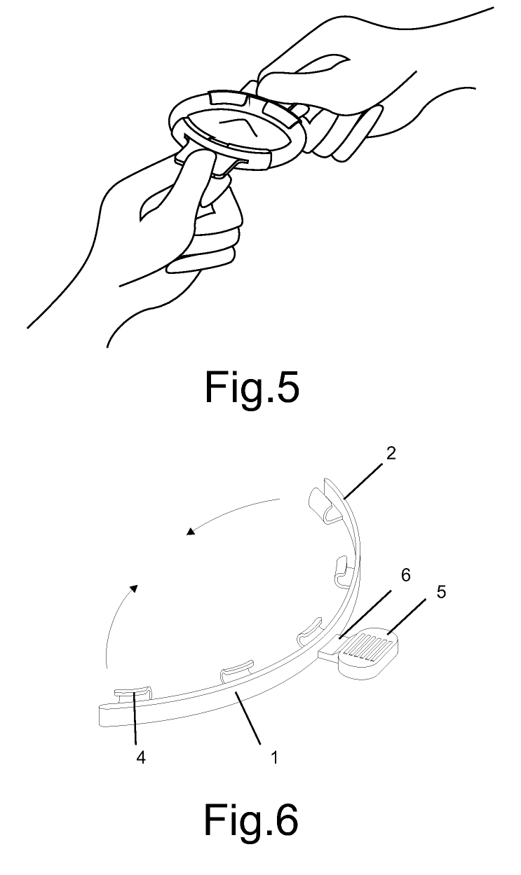

### DETAILED DESCRIPTION OF THE INVENTION

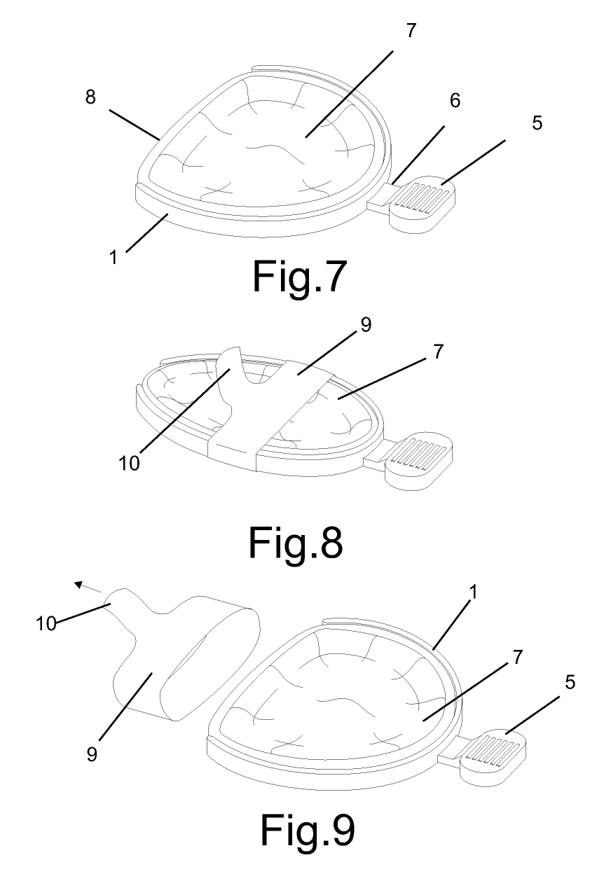

In FIGS. 6-7, one embodiment of the invention is represented. In this embodiment, a condom holder 1 includes an arc-shaped member 2 made of an elastic material. Fasteners 4 for holding a condom are formed on the inner side 3 of the member 2. The holder 1 is provided with a handle 5 connected to the holder by a crosspiece 6. The handle 5 is for holding the holder when putting the condom on.

When installing a condom 7 into the holder 1, the ends of the holder are brought together, and the edge 8 of the condom rolled into a ring is placed in the fasteners 4, as shown in FIG. 7. Elastic forces in the member 2 of the holder 1 are compensated by compressive forces of the edge 8 of the condom 7. The size, shape and elastic properties of the material of the member 2 are selected in such a way that the condom 7 would be stretched when the holder is in use, thus making the process of putting the condom on comfortable.

However, due to the fact that the quality of the condom may deteriorate when stretched for a long time, it is preferrable to hold the holder 1 with condom 7 in such a state that the area defined by the holder 1 is reduced and therefore, the condom ring is not stretched.

To ensure this, a ring 9 embracing the holder can be used, as shown in FIG. 8. The ring 9 is provided with a pull-tab 10 for its easy removal from the holder. After the ring 9 is removed, the holder is expanded due to the elastic forces, thus stretching the condom as shown in FIG. 9. The ring can be made of any material having sufficient strength, e.g. of plastic film.

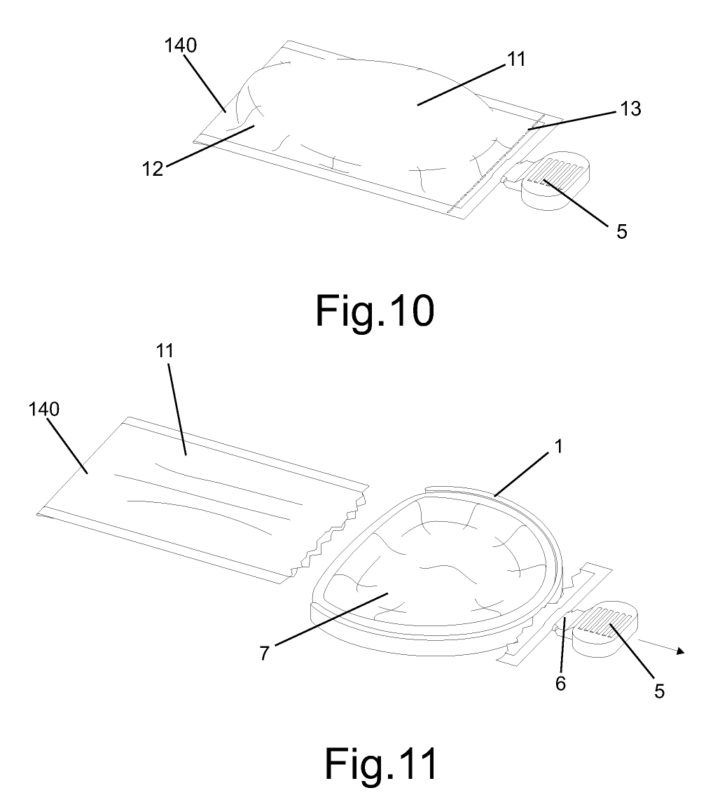

In another embodiment represented in FIGS. 10-11, a package 11 can be used as a retaining device. The package 11 is similar to a package used with traditional condoms, and is formed by a folded sheet 12 connected on the edges thereof.

The package 11 is preferrably made of polymer film and is provided with weakened lines, along which it can be easily torn. Said lines can be provided by forming the package of a two-layered film. One layer of the film (thin and solid) is impenetrable, and the other (more rigid) is provided in predetermined areas with weakened lines; to be torn therealong.

A preferable arrangement of a weakened line is shown in FIG. 10, in which a weakened line 13 runs along the package edge where the handle 5 of the holder comes out of package.

The size of the package 11 is selected in such way that the holder 1 placed therein with the condom 7 would remain in a state, in which the area defined by the holder 1 is reduced and thus the condom ring is not stretched.

The handle 5 of the holder 1 is outside the package 11. In the area where the crosspiece 6 protrudes out of the holder and therefore out of the package 11, the edges of the package 11 remain unconnected, embracing the crosspiece 6. To provide tightness, the connection between the package 11 and the crosspiece 6 can be tightened by glue or a sealing component (not shown in the drawings).

When unpacking the condom 7, the user holds the handle 5 is held with one hand, while pulling the edge 140 of the package 11 with the other hand. The package is therefore torn along the weakened line 13. After that, the package is removed and the holder is expanded due to the elastic forces of the member 2, thus stretching the condom 7 as shown in FIG. 11.

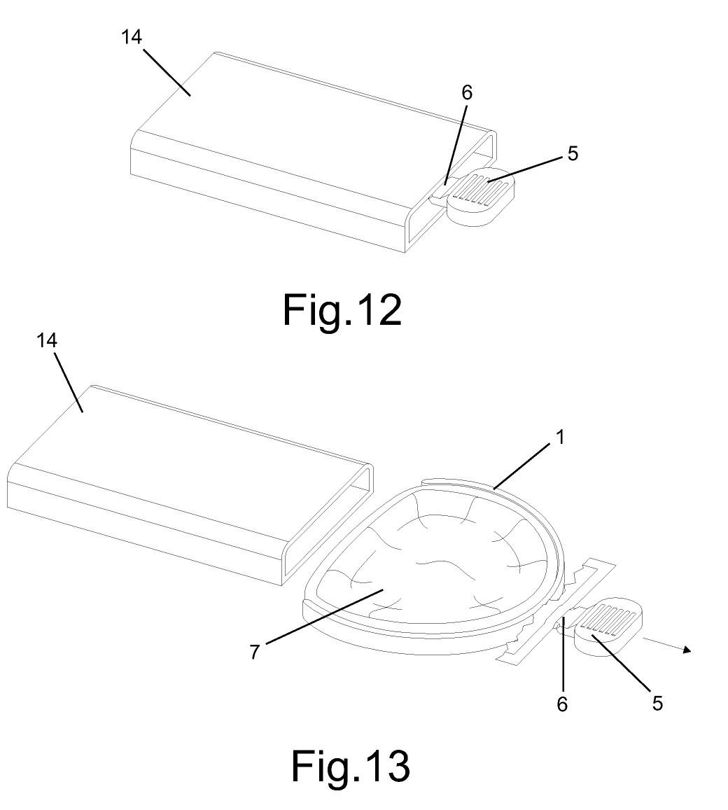

As shown in FIG. 12, the holder received in the package 11 can be placed in a case 14 such as a flat box with an open end 22. The box 14 can be made of any material having sufficient strength to ensure the integrity of its contents and allow holding it in hand, e.g. of plastic.

The package 11 received in the box 14 is bonded, e.g. glued, thereto. Connection points of the package 11 to the box 14 are preferably located on the edge of package 11 from which the crosspiece 6 protrudes, on both sides of the crosspiece (not shown in the drawings). The package however can be attached at longer areas, e.g. along this whole edge.

When unpacking the condom 7, the user holds the box 14 with one hand, while pulling the handle 5 with the other hand. The package is therefore torn and the holder 1 with the condom 7 placed therein comes out of the box 14 completely. After removing the box 14 and the package 11, the holder 1 is expanded, thus stretching the condom 7 as shown in FIG. 13.

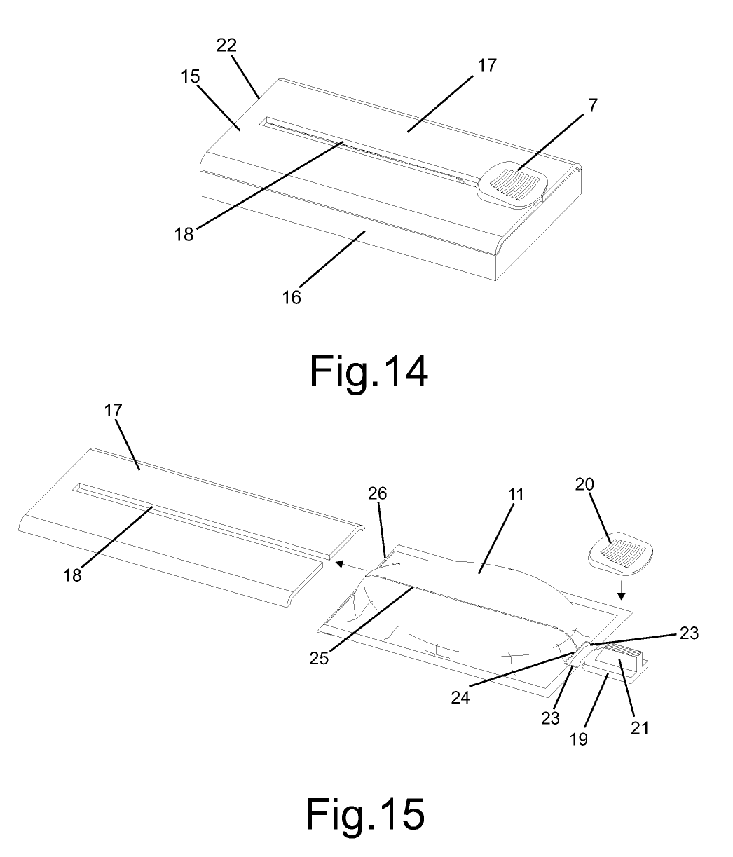

In another embodiment represented in FIGS. 14-17, the box 15 consists of two parts: the bottom 16 and the lid 17 connected to each other with lateral sides thereof. The lid 17 has a central through slot 18, beginning from one side of the lid and ending short of its other side. The connection between the bottom 16 and the lid 17 can be bonded.

A support 19 for a key 20 is attached to the outer edge of the holder 1. The support 19 is connected to the holder 1 by a crosspiece 6. The key 20 is a button or a key shaped and sized to be conveniently acted by a digit. The key 20 is connected to the support 19 by means of a leg 21 placed in a slot 18. In this embodiment, the holder 1, the crosspiece 6, the support 19, the leg 21 and the key 20 are integral. However, they can be made of different parts connected to each other.

The package 11 is provided with weakened lines 23-26 shown in FIG. 15.

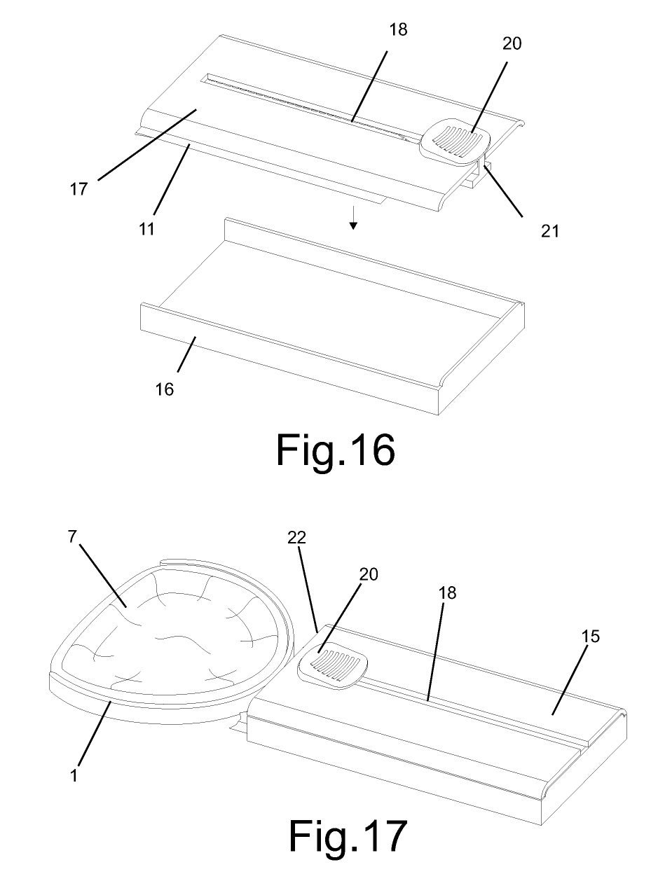

The process of packing the holder 1 with the condom 7 placed therein is shown in FIGS. 15-16. Firstly, the holder 1 received in the package 11 is connected to the lid 17 by placing the leg 21 into the slot 18. If necessary, the package 11 is fixed with respect to the lid 17. The whole unit is placed onto the bottom 16, and the lid 17 is fixed with respect to the bottom 16.

When unpacking the condom, the user holds the box 15 in one hand in such manner that the open end 22 is facing forward, away from the user, and acts on the key 20 with his thumb. As mentioned above, the holder 1 and the key 20 are integral, therefore the package 11 attached to the box 15 (as mentioned above) prevents the displacement of the key 20. Therefore, when the displacement of the key 20 begins, the front edge of the holder 1 thrusts against the front edge of the package 11. When the key 20 is displaced further, the frond edge of the package 11 is torn, and the holder 1 with the condom held therein moves out of package 11 and the box 15 through the open end 22 of the case. At the same time, the package 11 is torn at the opposite edge by the leg 21. When the key 20 is displaced to the end of the slot 18, the holder 1 goes out of the box 15 completely and is expanded by the elastic forces, thus stretching the condom 7 as shown in FIG. 17.

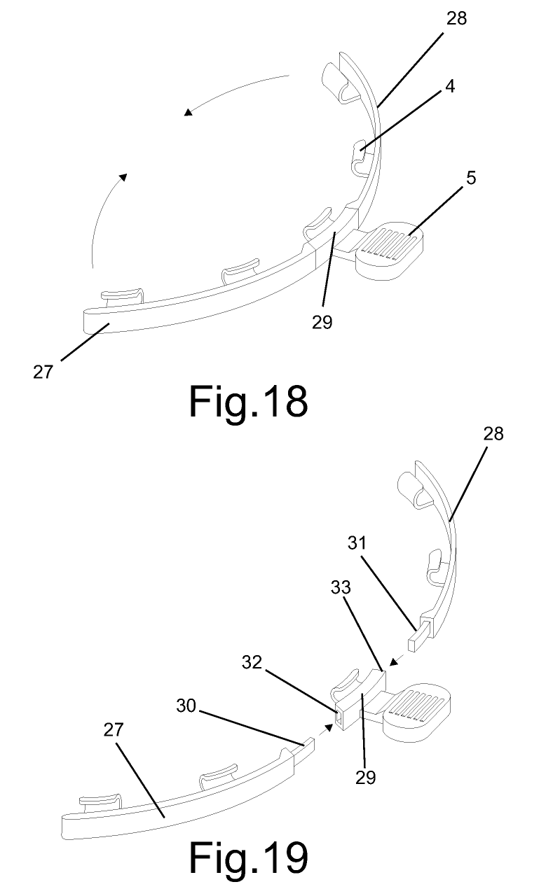

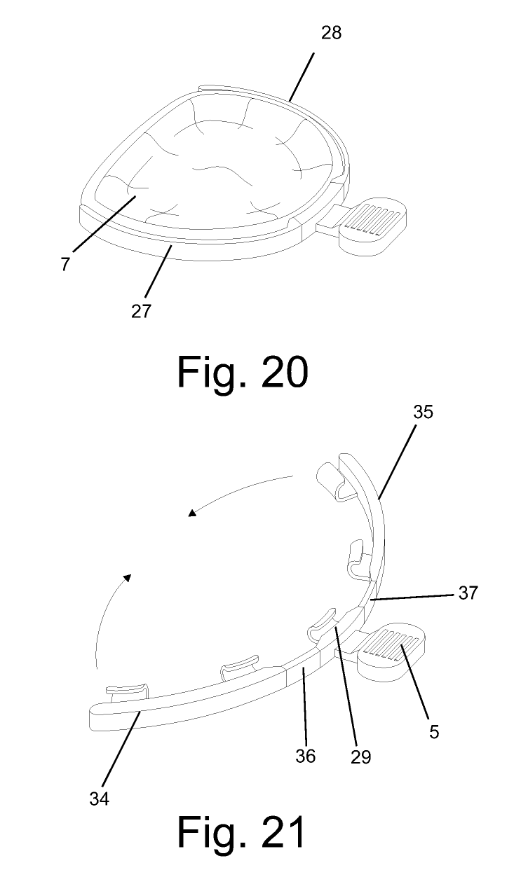

The holder can also consist of several parts, as shown in FIGS. 18, 19 and 20. In one embodiment (FIGS. 18-19) the holder includes two elastic arc members 27 and 28 connected by a connector 29 with a handle 5 attached to the outer side thereof. Fasteners 4 are located on the arc members 27, 28 and also on the connector 29.

Each arc 27 and 28 is provided with a lug 30 and 31, respectively. Sockets 32 and 33 are provided in the ends of the connector 29. Lugs 30 and 31 of the arc members 27 and 28 are tightly fixed in said sockets.

In the embodiment represented in FIG. 21, the holder includes rigid arc members 34, 35 connected to the connector 29 by elastic webs 36 and 37.

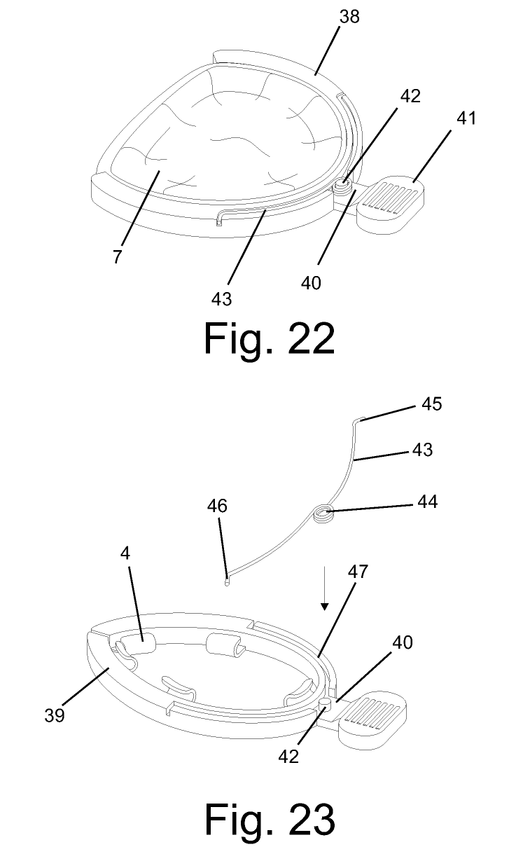

In yet another embodiment represented in FIGS. 22, 23, the holder 38 is a flat open ring 39 provided with fasteners 4 on the inner side thereof. A handle 41 is attached to the outer edge of the ring 39 by means of a crosspiece 40. An axis 42 is formed on the crosspiece 40. The holder is provided with a spring wire 43 which has coils 44 in its middle part, thus forming a torsion spring. This spring tends to bring the holder into a state where the space defined by the holder is expanded. Said coils 44 embrace the axis 42, and the ends 45, 46 of the wire 43 are fixed in the groove 47 on both sides of the crosspiece 40.

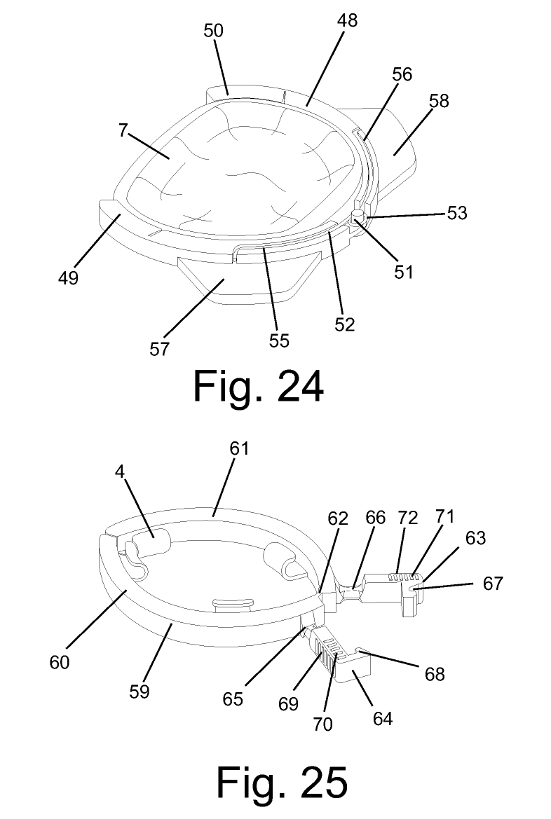

A holder 48 represented in FIG. 24, includes two arc members 49, 50 connected by a hinge 51 and provided with fasteners 4. Similarly to the previous embodiment (FIGS. 22-23), the holder 48 is provided with a spring wire 52 which has coils 53 in its middle part, the coils embracing the axis of the hinge 51. The ends of the wire 52 are fixed in grooves 55 and 56 in the arc members 49 and 50. The holder 48 also includes handles 57, 58 attached to the arc members 49, 50, respectively, on the outer side thereof.

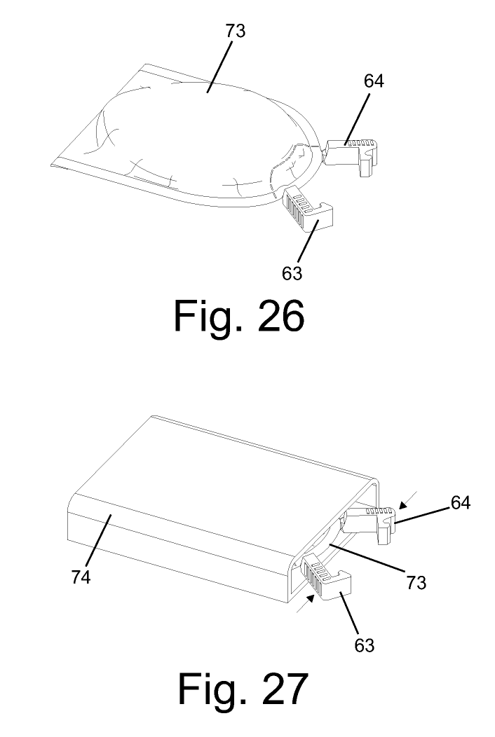

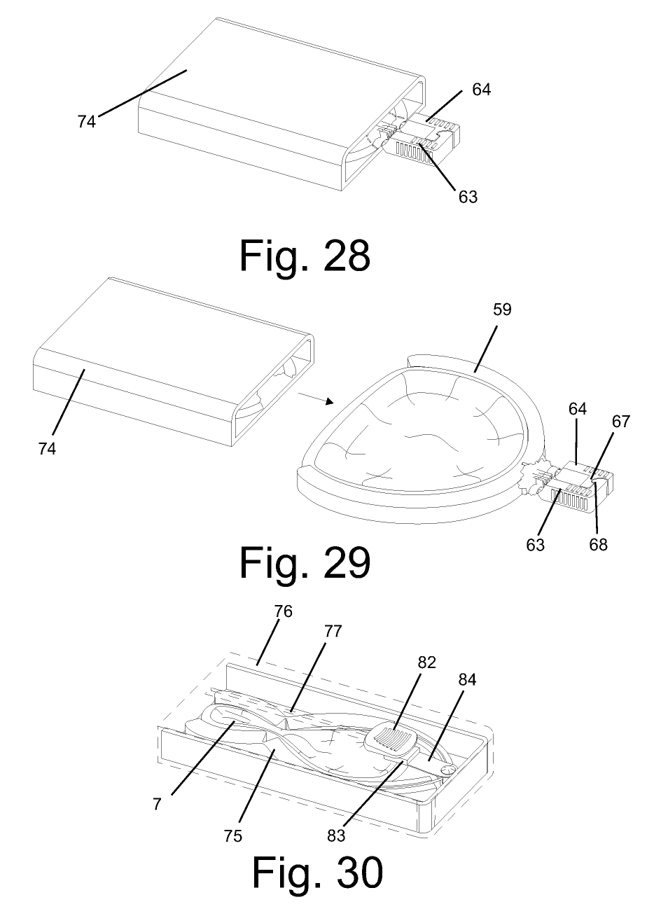

In the embodiment represented in FIGS. 25-29, the holder 59 includes two arcs 60 and 61 connected by an elastic web 62. Handles 63 and 64 are attached to the arc members 60 and 61 on the outer side thereof. Said handles are placed in close vicinity to the web 62 on two sides thereof and are connected to the arc members 60 and 61 by crosspieces 65 and 66. The handle 63 is provided with a recess 67 on the end thereof, and the handle 64 is provided with a lug 68 adapted to mechanically engage with the recess 67. By means of the recess 67 and lug 68, handles 63 and 64 can be fixed with respect to each other, and thus the holder 59 can be fixed in unfolded state (FIG. 29). Each handle 63, 64 has ribbed areas 69, 70 and 71, 72 on two adjacent sides thereof (FIG. 25). These areas facilitate gripping the handles with digits of the user's hand. The holder 59 can be received in a package 73 and case 74 (as described above with reference to FIGS. 10, 12), the handles 63, 64 extending outside the package 74 and case 74.

When unpacking the condom, the user holds the case 74 in one hand, while gripping the handles 63 and 64 with the other hand, thus bringing said handles together and fixing the lug 68 in the recess 67. Consequently, elastic forces appear in the holder 59 tending to unfold the holder, but suppressed by the package 73 and case 74. Further, the holder 59 is extracted from the case 74 while holding it by the handles 63 and 64. The package 73 attached to the case 74 is therefore torn and the holder 59 with condom comes out completely. The holder is unfolded due to the elastic forces, thus stretching the condom 7.

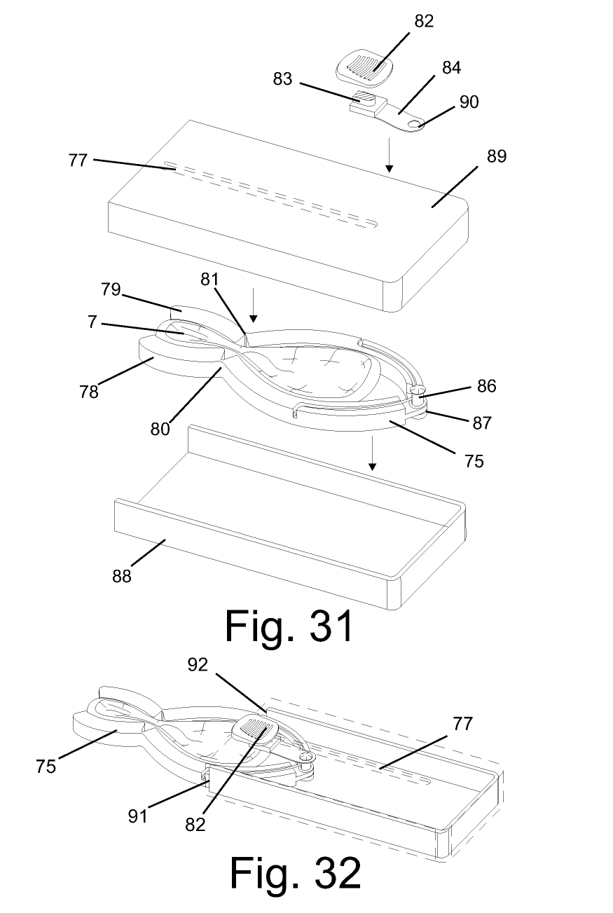

In yet another embodiment represented in FIGS. 30-35, a holder 75 is similar to the holder 48, represented in FIG. 24. The difference is that holder 75 is not provided with handles.

As shown in FIG. 30, the holder 75 with the condom 7 held therein is placed in a box 76, which consists of two parts: a bottom 88 and a lid 89 provided with a slot 77. Arc members 78, 79 of the holder 75 are provided with elastic webs 80, 81 which allow changing the shape of the holder. Due to this change of shape, a more compact arrangement of the holder in the box is achieved.

A key 82 is mounted outside the box 76 on a pin 83 which is received in the slot 77. The key 82 is connected to the holder by an elastic ribbon 84, one end of which is attached to the pin 83 and the other end has an opening 90 connectable with an axis 86 of a hinge 87 connecting the arc members 78, 79. The ends of the ribbon may be fixed by any known means which provide sufficient durability.

When the key 82 is displaced, the ribbon 84 is pulled and pulls the holder 75 forward.

In contrast to the embodiment represented in FIGS. 14-17, in which the displacement of the holder to the end of the slot results in complete extraction of the holder, here the displacement of the key to the end of slot 77 leads to the transition of the holder into a position in which the holder can go out of the box automatically due to the elastic forces applied by the spring and to the interaction of the holder arc members with the walls 91, 92 on the open end of the box. In FIG. 34 the relative position of the key and the holder is shown where the key has reached the end of the slot. In FIG. 35, the holder has come out of the package. This type of connection between the holder and the key makes extraction of the holder from the box smooth.

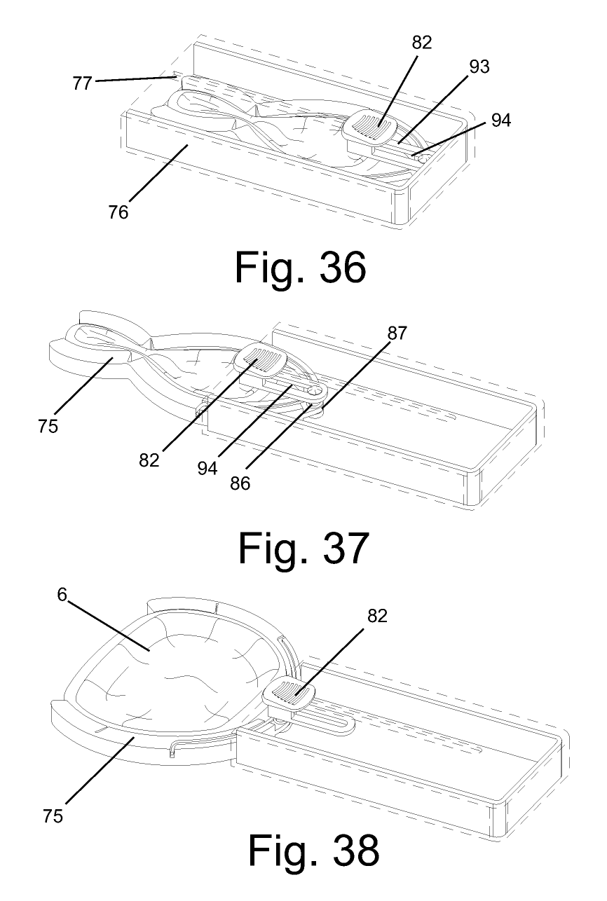

In the embodiment represented in FIGS. 36-40, in contrast to the embodiment represented in FIGS. 30-35, here the ribbon 84 is replaced by a bracket 93 attached to the pin 83. The bracket 93 includes a guide slot 94 in which the axis 86 of the hinge 87 is moveably received.

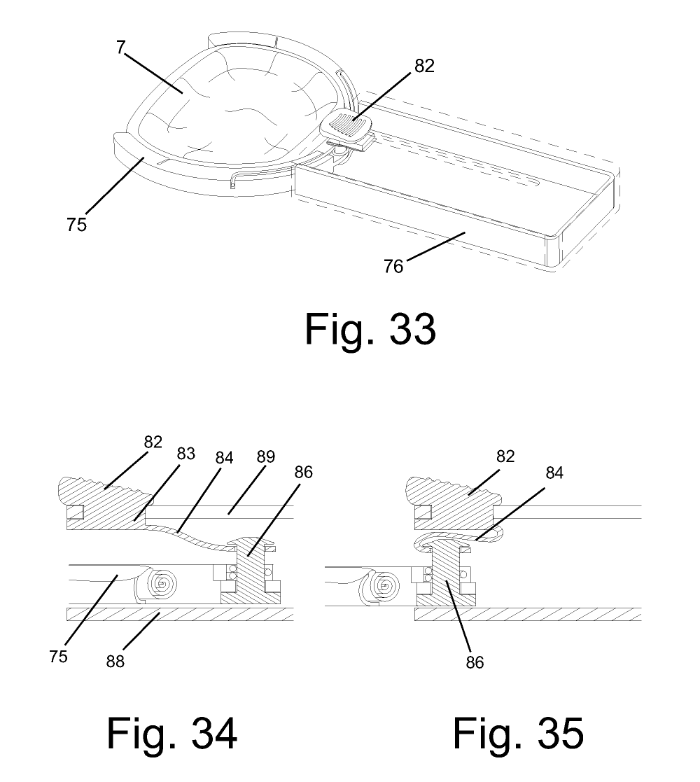

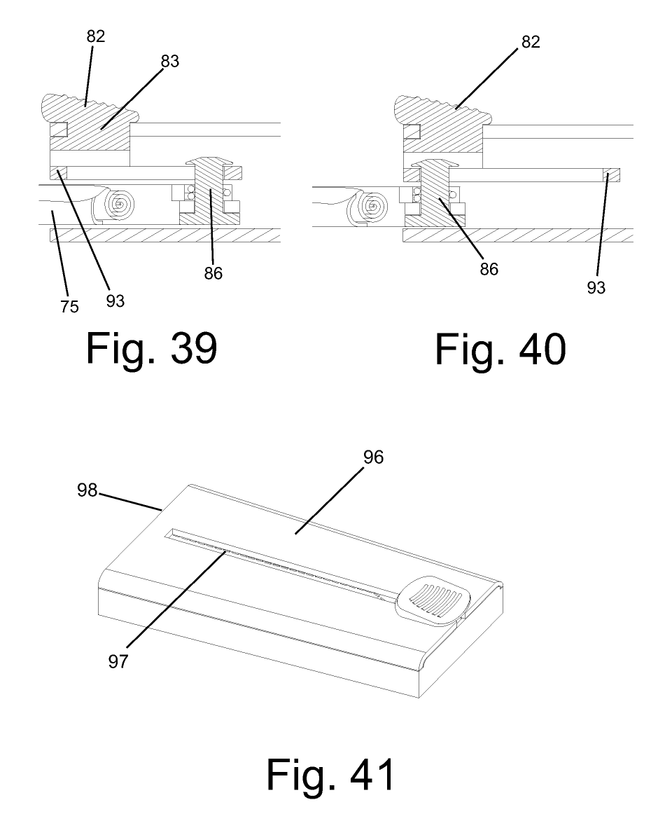

The unpacking process shown in FIGS. 37, 38, is similar to that shown in FIGS. 32, 33. When the key 82 reaches the end point, the holder 75 is displaced on its own; the axis 86 of the hinge is displaced in the slot 94. In FIG. 39, the relative position of the key and the holder is shown at the moment when the key has reached the end of the slot. In FIG. 40, the holder has come out from the package.

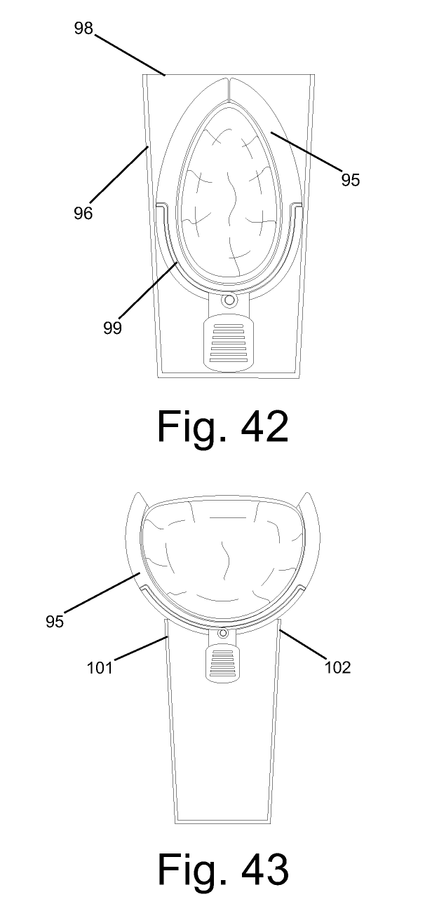

An embodiment is also possible, in which a holder 95 similar to the holder shown in FIG. 22, is placed in the box 96 which is widened towards its open end 98, as shown in FIGS. 41-43.

This design of the box (as well as two previous examples shown in FIGS. 30-40) provides make extraction of the holder 95 from the box 96 smooth due to the action of the elastic forces of a spring 99 and due to interaction of the holder 95 with walls 101 and 102 of the box at the open end 98.

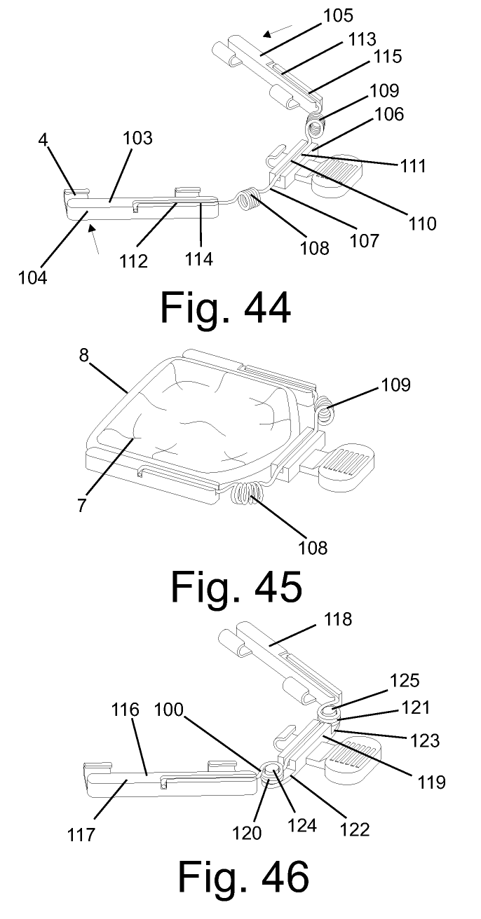

Other embodiments of the holder are also possible, shown in FIGS. 44-49, in which the holder is adapted to be expanded due to the elastic properties of the spring.

A holder 103 represented in FIGS. 44-45, is formed by three elongated members 104, 105 and 106 connected to each other by a spring wire 107 and provided with fasteners 4.

The wire 107 has coils 108 and 109 placed between the frames 104 and 106, 105 and 106. The middle part 110 of the wire is fixed in a groove 111 formed in the member 106, and the ends 112 and 113 of the wire are fixed in grooves 114 and 115 formed in the members 104 and 105, respectively.

When installing the holder 103 of the condom 7 in the fasteners 7, the members 104 and 105 are displaced towards each other, coils 108 and 109 are therefore deformed and the elastic forces appearing in said coils are balanced by the compression forces of the edge 8 of the condom 7.

In the embodiment represented in FIG. 46, the holder 116 is also formed by three members 117, 118 and 119 connected to each other by a spring wire 100. However, in contrast to the previous embodiment (FIGS. 44, 45), here the coils 120 and 121 form torsion springs, and the ends of the member 119 is formed with lugs 122 and 123 provided with axes 124 and 125. The coils 120 and 121 embrace said axes 124 and 125.

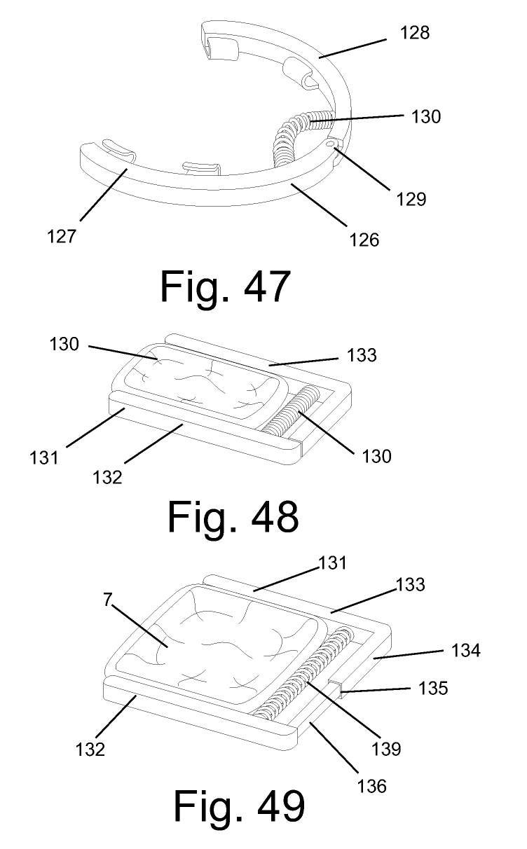

A holder 126 represented in FIG. 47, is formed by two semicircular members 127 and 128 connected to each other by a hinge 129. An extension spring 130 is attached to said members on their inner side.

In yet another embodiment represented in FIGS. 48, 49, a holder 131 is formed by two L-shaped members 132 and 133. Part 134 of the member 133 is hollow and has a socket 135, and part 136 of the member 132 is moveably received in this socket. Parts 136 and 134 of the members 132 and 133 are connected by a compression spring 139 which keeps the holder 131 in a state where the space defined by the holder is expanded, as shown in FIG. 49.

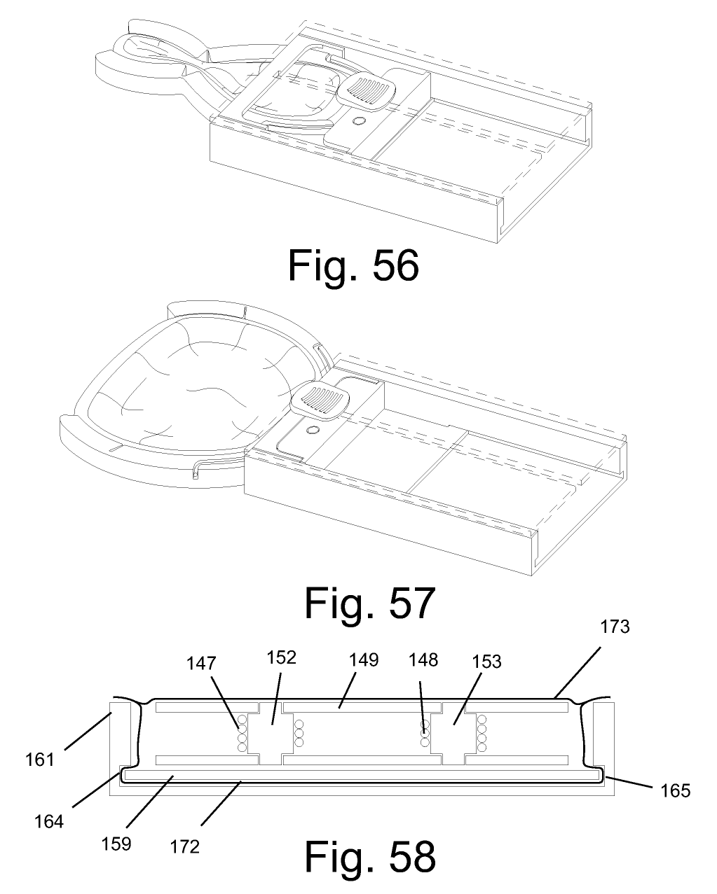

In FIGS. 50-58, another embodiment is represented, where a holder 141 includes two arc members 142 and 143 with elastic webs 154 and 155 formed on the outer side thereof. The arc members are connected by a spring wire 144. The ends of the wire 144 are fixed in the grooves 145, 146 formed in the arc members 142, 143. As shown in FIG. 50, coils 147 and 148 are formed in the middle part of the wire, forming torsion springs. Said coils are connected to a connector 149 having a U-shaped cross-section. In the recess of the connector 149 the coils 147 and 148 are positioned. Coils 147 and 148 surround pins 152 and 153. The connector 149 has openings 150 and 151 in which the ends of the pins 152 and 153 are received. Lugs 156 and 157 are formed on the ends of the connector 149. A leg 167 for supporting the key 168 therein is formed on one side of the connector 149 in its middle part.

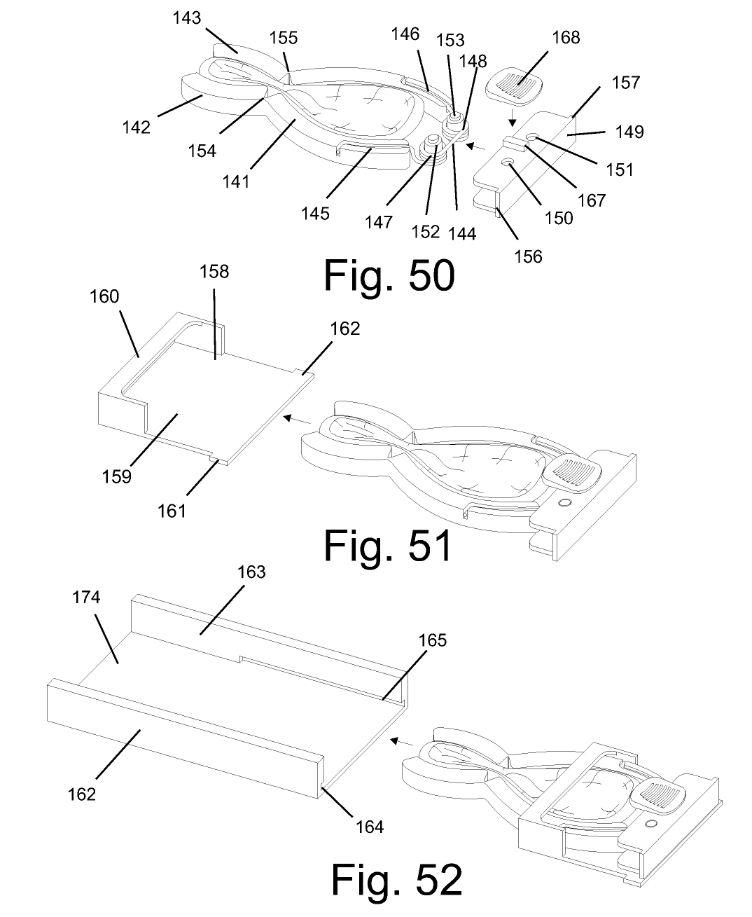

The holder 141 is held in folded state by means of a retaining member 158 comprising a plate 159. On this plate, the holder 141 with connector 149 is placed as shown in FIGS. 51-52). The retaining member 158 further comprises a frame 160 on one end of the plate 159, the a frame 160 embracing the folded holder 141 in the likeness of the ring 9 shown in FIG. 8. Lugs 161 and 162 are formed at the other end of the plate 159.

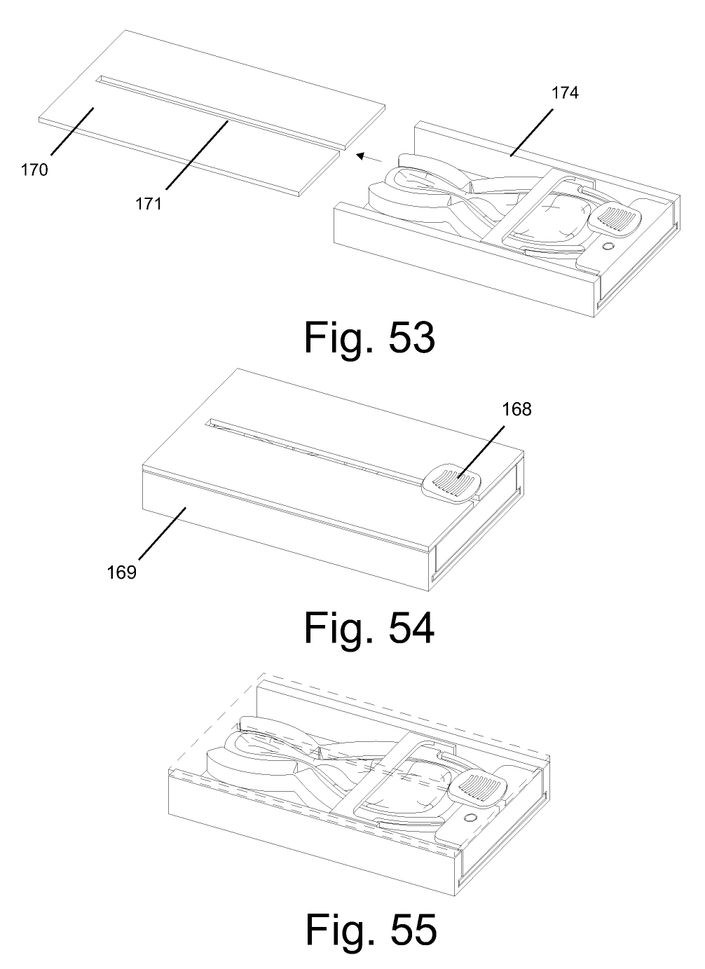

The retaining member 158 mounted over the holder 141 is received in a package formed by two sheets 172 and 173 connected at the edges thereof, as shown in FIG. 58, and is placed into a flat box 169 formed by two parts: a bottom 174 and a lid 170 with a central through slot 171 (FIGS. 53, 54). The bottom 174 is formed by a plate defined on its two sides by flanges 162 and 163. Grooves 164 and 165 are formed along the flanges 162 and 163 near the base thereof (FIG. 52) for the lugs 161 and 162 of the plate 159 to be guided in said grooves.

When packing the condom, the sheet 172 is placed into the bottom 174 in such a way that the sheet 172 follows the inner periphery of the bottom including the grooves 164, 165. Further, the sheet is bonded to the bottom. Further, the retaining member 158 with the holder 141 is placed in the bottom by inserting the lugs 161 and 162 into the grooves 164 and 165. The sheet 173 is attached on top and its edges are connected with the edges of the sheet 172, as shown in FIG. 58. The lid 170 is put on the whole assembly by inserting the pin 167 into the slot 171.

When unpacking the condom, the user presses on the key 168, displacing it away from him while holding the box 169 in one hand. Subsequently, the holder 141 with the retaining member 158 starts coming out of the box. When the lugs 161 and 162 thrust against the ends of the grooves 164 and 165, the retaining member 158 stops, while the holder keeps displacing forward. When leaving the box, the holder 141 is unfolded by the force of the spring and thus stretches the condom, as shown in FIG. 57.

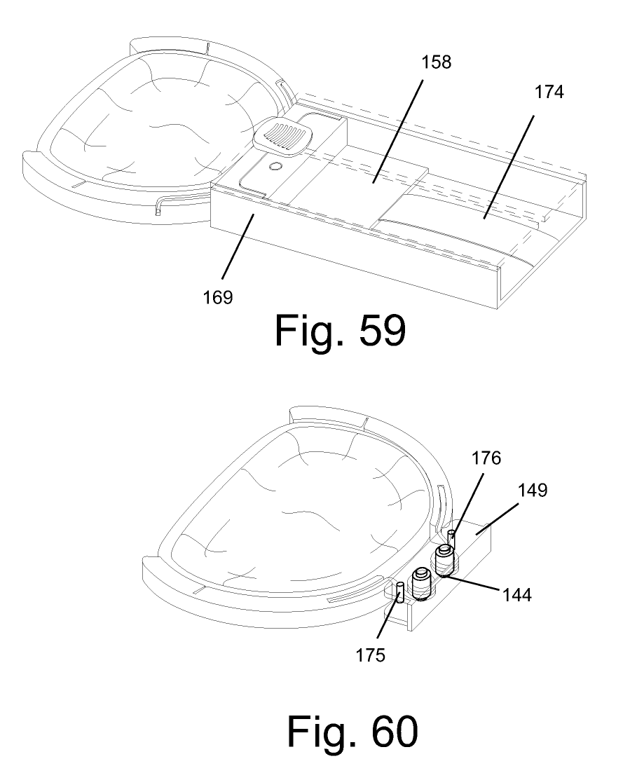

It is clear that the means preventing the retaining member 158 from leaving the box 169 (in the embodiment described above said means are represented by the grooves 164 and 164) can be realized in many different ways. E.g., the retaining member 158 can be connected to the bottom of the box 169 by a flexible ribbon 174 which keeps the retaining member 158 inside the box when unpacking the condom, as shown in FIG. 59.

In some embodiments not shown in the drawings, the retaining member may come out of the box completely and is then removed by the user. However, it is preferred that when unpacking the retaining member remains inside the holder after reaching the open end of the box and being fixed in that position, as shown in FIGS. 50-58.

Furthermore, the holder 141 can be provided with stops preventing the excessive twisting of the spring, which may lead to damaging the condom or to excessive friction between the condom and the holder. As shown in FIG. 60, the stops can be pins 175 and 176 located on the connector 149, the pins retaining the wire 144 in position. Therefore, there is no need to select the spring properties precisely to provide the appropriate degree of stretch of the condom.

In some embodiments not shown in the drawings the spring can provide only a part of the effort required to stretch the condom to a desired degree ( ). The rest of the required effort can be provided by the user. Therefore, the spring only facilitates the stretching of the holder.

Furthermore, in the described examples, the condom holder is provided with fasteners formed as hooks. However, other types of fasteners for fixing the edge of a condom can be used, e.g., fasteners formed as a pair of rollers. Further, the holder can be an open ring with a shaped groove extending along the inner circumference thereof. If the holder is used with a microcondom, the latter can be fixed to the holder by means of a light adhesive.

Within the scope of the appended claims, the invention can be implemented in many embodiments other than those described herein.

I claim:

1. A condom holder comprising fasteners for fixing the edge of a condom, and a connection between these fasteners; said connection at least partially being elastic, the holder at least partially defining a space in which the condom can be placed; the holder being adapted to change the shape of said connection between a first configuration in which said space is reduced, and a second configuration in which said space is expanded, characterized in that in the first said configuration at least part of said connection is strained and tends to transform into the second configuration, and in the second configuration said strain at least partially is released, the holder being provided with a fixing device for fixing the holder in the first configuration.

2. A holder according to claim 1, characterized in that the connection includes at least one arc-shaped member.

3. A holder according to claim 2, characterized in that the connection includes at least one more arc-shaped member and at least one connector connecting the arc-shaped members.

4. A holder according to any one of claims 32, characterized in that the arc-shaped member is elastic.

5. A holder according to claim 3, characterized in that the connector is elastic.

6. A holder according to claim 1, characterized in that in the second configuration the distance between the fasteners is larger than the distance between the fasteners in the first configuration.

7. A holder according to claim 2, characterized in that in the second configuration the distance between the ends of the arc-shaped member is larger than the distance between the ends of the arc-shaped member in the first configuration.

8. A holder according to claim 1, characterized in that said ability to change the shape is provided by at least one spring.

9. A holder according to claim 8, characterized in that the spring comprises an elastic wire.

10. A holder according to claim 9, characterized in that the spring is formed with coils in the middle part thereof.

11. A holder according to claim 10, characterized in that the holder includes an axis disposed in the middle part of the holder, and the spring is mounted on said axis with its coils embracing the axis.

12. A holder according to claim 9, characterized in that it includes grooves in which both ends of the spring are placed.

13. A holder according to claim 8, characterized in that the spring is a compression spring.

14. A holder according to claim 8, characterized in that the spring is an extension spring.

15. A holder according to claim 8, characterized in that the spring is a flexure spring.

16. A holder according to claim 8, characterized in that the spring is a torsion spring.

17. A holder according to claim 3, characterized in that said ability of changing the shape of the connection between the fasteners is provided by at least one elastic connector connecting the arc-shaped members.

18. A holder according to claim 3, characterized in that said ability of changing the shape of the connection between the fasteners is provided by a hinge connecting the arc-shaped members.

19. A holder according to claim 3, characterized in that said ability of changing the shape of the connection between the fasteners is provided by a hinge connecting the arc-shaped members and by elastic webs in the arc-shaped members.

20. A holder according to claim 2, characterized in that the holder is provided with a handle on the outer side of the arc-shaped member.

21. A holder according to claim 20, characterized in that the holder is provided with another handle, both handles being arranged in such a way that when the handles are acted, the shape of the holder is changed.

22. A holder according to claim 21, characterized in that the handles are provided with a fixing device for fixing the holder in the second configuration.

23. A holder according to claim 1, characterized in that the holder is provided with a retaining device for retaining the holder in the first configuration.

24. A holder according to claim 23, characterized in that the retaining device for retaining the holder in the first configuration includes a ring embracing the holder.

25. A holder according to claim 23, characterized in that the holder is at least partially received in a package.

26. A holder according to claim 25, characterized in that the package is provided with at least one weakened line.

27. A holder according to claim 25, characterized in that the holder is provided with a case into which the package with the holder received therein is placed, the package being attached to the case.

28. A holder according to claim 23, characterized in that the retaining device for retaining the holder in the first configuration is a box having one open end, wherein the holder is received in the box.

29. A holder according to claim 28, characterized in that the box includes a slot and the holder is provided with a key connected to the holder by means of a part received in said slot and adapted to displace, wherein said key is outside the box.

30. A holder according to claim 1, characterized in that the holder is provided with a retaining device for retaining the holder in the first configuration and a fixing device for fixing the holder in the second configuration.

31. A holder according to claim 31, characterized in that the holder is received in a box with a slot, the retaining device for retaining the holder in the first configuration includes a ring embracing the holder, and the holder is provided with a key connected to the holder by means of a part moveably received in said slot; wherein the key is outside the box.

32. A holder according to claim 32, characterized in that said ring is moveably connected to the box and provided with a device preventing the ring from leaving the box when displacing the holder in the condom plane.

33. A condom holder for holding a flatly arranged condom, the holder including fasteners for fixing the condom edge, and a connection between these fasteners; said connection at least partially being elastic, the holder at least partially defining a space in which the condom is placed; the holder being adapted to deform between a first configuration in which said space is reduced, and a second configuration in which said space is expanded, characterized in that in the first said configuration at least part of said connection is strained and tends to transform into the second configuration, and in the second configuration the condom is stretched, and said strain is partially released and balanced by elastic forces resulting from the stretch of the condom, the holder being provided with a fixing device for fixing the holder in the first configuration.
